(19) United States
(12) Patent Application Publication
(10) Pub. No.: US 2002/0150935 A1
(43) Pub. Date: Oct. 17, 2002

(54) Dotted micro-array data extraction method

(76) Inventors: Zheng-Zheng Zhou, Houston, TX (US); Jaime A. Stein, Sugar Land, TX (US); Qien Zhou Ji, Sugar Land, TX (US)

Correspondence Address: Roger Fulghum, Baker Botts L.L.P., One Shell Plaza, 910 Louisiana Street, Houston, TX, 77002-4995, US

(21) Appl. No.: 10/043,686

(22) Filed: Jan. 11, 2002

Related U.S. Application Data
(60) Provisional application No. 60/261,305, filed on Jan. 12, 2001.

(51) Int. Cl. C12Q 1/68; G01N 33/53; G01N 33/542; G06F 19/00; G01N 33/48; G01N 33/50

(52) U.S. Cl. 435/6; 702/19; 435/7.9

(57) ABSTRACT

An automated method for identifying the precise location of each sub-array and dot of a micro-array is provided. The sub-arrays of the micro-array are located using a template sub-array. The estimated location of each sub-array is estimated according to the expected physical dimension of the sub-array. A search of each sub-array is conducted for the dots of each sub-array. A constraining shape mask is constructed, and a segmentation method is applied to detect the location of each dot within the collection of pixels. The constraining shape mask is applied to each detected dot, limiting the boundary of and precisely identifying the location of each dot.

### CROSS-REFERENCE TO RELATED APPLICATIONS

The application claims the benefit of provisional U.S. Application Serial No. 60/261,305, filed Jan. 12, 2001, and titled “GLEAMS: A Novel Approach to High Throughput Genetic Micro-Array Image Capture and Analysis,” which is incorporated herein by reference in its entirety.

### BACKGROUND OF THE INVENTION

Many members of the scientific community believe that genetic information lies at the root of many diseases, and that genes are likely to be responsible for these diseases, whether the genes are the causes of disease, or otherwise promote disease through the encoding of proteins. The Human Genome Project has provided significant information on the recipes for proteins. The Human Genome Project has enhanced the ability to diagnose, if not eventually cure, many diseases.

It is also well known that knowledge of the raw sequence of the more than three million base pairs in the human genome is insufficient in and of itself to permit the diagnosis or cure of any disease. Identifying genes requires the mapping of the genome into chromosomes and the identification of exons and introns. Identifying genes, however, is only the beginning; an analysis of the role, purpose and position of a gene within a pathway should facilitate an understanding of the causal relationship between genes, the illness, and the stage of the development of the illness.

As part of the development of the human genome projects, scientists have recognized the concept of differential gene expression. Gene expression is the process by which the coded expression of a gene is converted into the structures that are present and operating in the cell. The first step in gene expression is transcription, which is the process in which mRNA is formed by RNA polymerase to be complementary to a DNA sequence. Following transcription, mRNA serves as a template for protein synthesis through a process called translation. The expression level of a gene, a measure of its level of activity in a cell line, correlates with the density of its corresponding mRNA. The expression level of any particular gene can vary from individual to individual and vary with the progression of a disease or a treatment in a single individual. The knowledge of an expression level of a gene therefore assists a researcher in determining whether that gene responds to a trait, stimulus, or disease.

Genes can also be investigated at the DNA level, which, as described above, creates a complementary template for the transcribed gene. Various types of genetic alterations can be seen at the DNA level. The complementary DNA template may contain an alteration in the complementary template region, thus producing altered mRNA. In addition, an alteration may occur outside the coding region, creating an error during transcription or translation. Finally, the human genome is assumed to have many variable or polymorphic sites known as single nucleotide polymorphisms (SNPs). Scientists are currently attempting to correlate disease with a set of SNPs in genomic DNA.

The ability to construct micro-arrays from cDNA, hybridize them with control and treatment samples, and analyze them, opens the door to comparative research and analysis. Gene micro-array technology enables researchers, for example, to measure simultaneously the expression levels of a large number of genes in a normal tissue sample relative to a cancerous tissue sample. By inspecting the gene expression on a micro-array for tissue samples of, say, melanomatic and healthy skins, it is possible to identify a group of genes responsible or at least related to the disease. Thus, gene micro-array technology enables the simultaneous measurement of gene expression levels in a normal tissue sample relative to a test condition, such as, for example, cancerous tissue.

One of the goals of a micro-array experiment is to simultaneously examine the expression of all genes of a specific organism in a cell type in a specific growth or stress condition. Micro-array technology requires the positioning of minute amounts of, for example, DNA representing specific genes, onto a small glass slide in an ordered fashion. This produces the DNA micro-array, which can contain, depending on the availability of clones from an organism, a specific subset of genes, or all genes. Thus, with a single experiment, gene array technology allows researchers to measure simultaneously a large number of genes in a tissue sample under normal conditions and under test conditions. Micro-arrays can be made by placing hundreds or even thousands of genes on a glass or nylon substrate. The cDNA material for each gene is deposited as a small dot onto the array. Because the identity of the cDNA at a particular element in the array is known, the identification of expressed genes simply follows a determination of which elements in the array have formed a binding complex with the sample DNA placed on the array.

In the course of a micro-array experiment, mRNA from two tissue samples—one control sample and the other a test sample—are each labeled with a fluorophore. If a single array is used, each sample receives a spectrally distinct fluorophore. For example, the control sample may be labeled with a fluorophore that emits in the green region, and the test sample may be labeled with a fluorophore that emits in the red region. Likewise, if only a single fluorophore is used for labeling, then multiple arrays—one for the test sample and one for the control sample—are required. The labeled samples are hybridized with the micro-array. Following hybridization, the arrays are washed at a chosen stringency to maximize the amount of perfectly complementary material bound to the micro-array, and, at the same time, minimize the amount of mismatched material bound to the array. Following washing, the micro-arrays are scanned with lasers that excite one or more of the fluorophores. The resulting fluorescence is captured, and the two fluorescent images are combined to produce a single color image or to generate two gray-scale images, one for each tissue sample. The two images can also be considered as two channels of a color image, which is known as a two-color fluorescence image. The experiment described above can also be done with a single tissue sample. In either case, the intensity of a captured fluorescence at each element in the micro-array correlates to the expression level of the corresponding gene in the tissue used to produce the resulting image.

In recent years, micro-array technology has become a very important tool in the analysis of gene expression and SNP studies. The development and use of micro-array technology has exploded in the past few years. Improvements have occurred in the areas of hardware, biological assays, and data quality. The rate of growth of micro-array use has increased, creating an ever increasing mound of data for analysis. Associating each micro-array element with a known grid location is currently an inefficient task. For example, this process typically requires manually locating each dot in the micro-array and then determining the coordinate location in the x and y direction for every element in the micro-array. The process of selecting, identifying, and providing a coordinate location for every element in the micro-array becomes unmanageable when there are hundreds or thousands of elements in the micro-array and when there are dozens or hundreds of micro-arrays. The greatest challenge is to analyze large amounts of data in an automated, high-throughput fashion. Any such analysis system will require a computer, digital signal processing (DSP), and analytical tools. Few integrated systems, and even less software, is available to solve this problem.

### SUMMARY OF THE INVENTION

The invention disclosed herein is an automated method for locating the sub-arrays of a micro-arrays and for locating within each sub-array the dots of each sub-array. For each micro-array image, the orientation and the lattice constant of each sub-array is calculated. After the orientation of the sub-arrays are determined, the sub-arrays may be rotated so that each sub-array is aligned with the rectilinear direction of the rows and columns of the micro-array. Regions of the micro-array image are compared to sub-array profiles to identify the sub-array regions of the micro-array. Once the location of each sub-array is determined, an estimate of the location of each dot is determined on the basis of the lattice constant.

A search of each sub-array is conducted to locate the dots of each sub-array. As part of this search, objects are located that are approximately round in size and are a collection of several pixels. Once the dots of the sub-arrays have been located, a two-dimensional vector field is constructed. The vector fields represents a two-dimensional displacement value of actual dot placement, as shown by the search for dot-like collections of pixels, versus the estimated dot placement. From these displacement values, an estimate of the position of each dot can be calculated. A constraining shape mask is constructed, and a segmentation method is applied to detect the location of each dot within the collection of pixels. The constraining shape mask is applied to each detected dot, limiting the boundary of and precisely identifying the location of each dot.

A technical advantage of the disclosed invention is an automated process that precisely identifies the location and boundary of each dot of each sub-array of a micro-array. Because of the method disclosed herein, a previously labor-intensive task of identifying each sub-array and dot has been simplified through automation such that hundreds of sub-array images may be processed through the method disclosed herein. Another technical advantage of the present invention is a method in which the process of locating each sub-array and dot is conducted with some degree of mathematical precision. Rather than employing a manual and time-consuming process of the individual location of sub-arrays and dots within, the method disclosed herein accomplishes this process according to a precise, automated method.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIG. 1 is a graphical representation of a micro-array;

FIG. 2 is a flow diagram of method steps for orienting a micro-array image and locating the sub-arrays of a micro-array;

FIG. 3 is a flow diagram of method steps for estimating the position of each dot of each sub-array; and

FIG. 4 is a flow diagram of method steps for estimating the detecting and delineating the position of each dot of each sub-array.

### DETAILED DESCRIPTION OF THE INVENTION

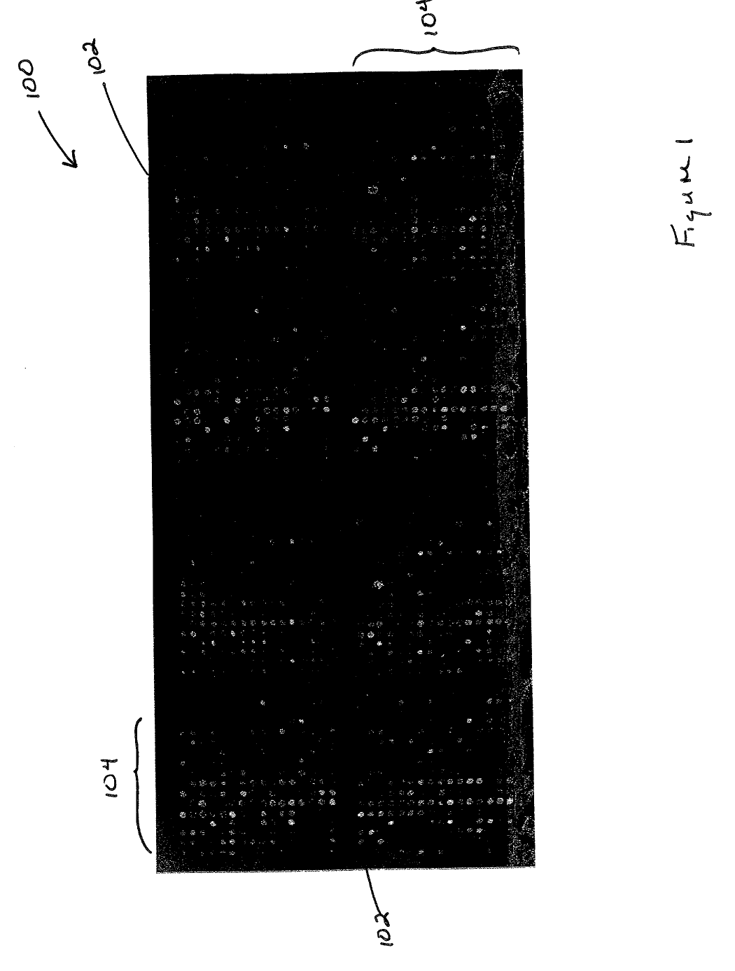

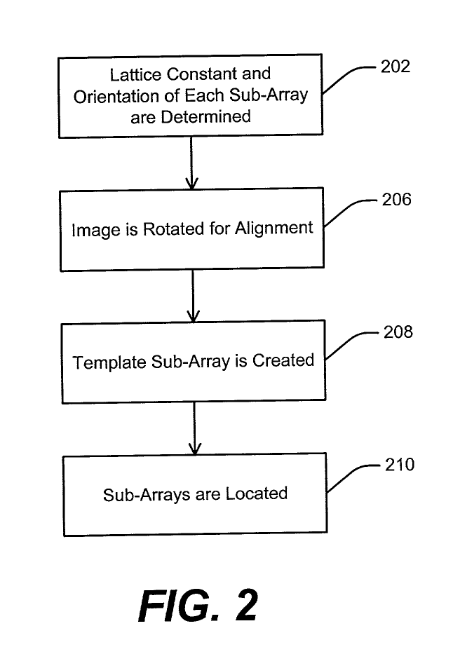

Shown in FIG. 2 is a graphical representation of a micro-array 100. In a typical micro-array, the dots 102 are organized into one or more sub-arrays, which are shown in FIG. 2. The sub-arrays 104 are arranged in an arrangement of four columns by two rows. In the example of FIG. 1, the dots of each sub-array are arranged in a rectangular grid of four columns and five rows. Often, sub-arrays are themselves arranged in a rectilinear fashion to form the micro-array. The most easily obtained and often the only reliable information about the image of a micro-array, before it is visually examined, is the spotting geometry of the micro-array. The spotting geometry of the micro-array is the number of rows and columns of the dots in each sub-array and the number of rows and columns of the sub-arrays. The method described herein uses as inputs the spotting geometry, together with the images themselves. The method described herein does not assume the size of the dot of a micro-array and does not assume that the distance between neighboring dots is known. Similarly, the method described herein does not assume that every dot in each sub-array is present or that the grids formed by the dots are highly regular. The method described herein is able to process a large number of micro-array images because the method involves a top-down approach of discerning the sub-arrays before detecting and analyzing the dots of the sub-arrays.

The method described herein involves orienting the micro-array image and locating the sub-arrays of the micro-array, an optional step of refining the dot placements within the micro-arrays, and delineating the extent of each dot. With respect to the step of orienting the micro-array image and locating the sub-arrays of the image, an automated method detects the orientation of the array grid and locates the sub-arrays within the image. With respect to the optional step of refining the dot placements within the micro-arrays, the grid points within each sub-array are slightly adjusted to account for any deviation from regularity in the sub-arrays. With respect to the step of delineating the extent of each dot, a threshold-based object detection method followed by an optional shape manipulation step is employed to delineate the boundary of each dot, thereby separating signal pixels from background noise or other signals.

### I. Image Orientation and Location of Sub-Arrays

A flow diagram of the method steps for spatially orienting the micro-array image and locating the sub-arrays of the micro-array is shown in FIG. 2. With respect to the step of orienting the micro-array image and locating the sub-arrays of the micro-array, the dimensions of the sub-arrays are estimated. Following an estimation of the dimensions of the sub-arrays, a template sub-array, representing a sub-array having a set of identical dots perfectly positioned in the sub-array is used to detect the actual location of the sub-arrays. At step 202, the lattice constant and the orientation of the sub-arrays are determined. If the number of rows and columns of a sub-array are known, the size of the sub-array can be determined by the lattice constant, which is the average distance between centers of neighboring dots. If it is the case that the location of none of the dots of a sub-array are known, the lattice constant can be estimated by examining the periodicity of structures within the image.

With respect to examining the periodicity of structures to determine a lattice constant, an approach for determining the lattice constant is to identify peaks in a 2-D periodogram of the image. Within the 2-D periodogram, the lattice constant can be determined by measuring the distance between the two strongest peaks of the periodogram. Using this approach, the orientation of, i.e. the angle formed by, a vector from the coordinate origin to the closest peak provides the angle of rotation of the grid array. Any noise introduced by false peaks can be at least partially overcome by using an averaged periodogram, which can be calculated by dividing the image into overlapping blocks and averaging the periodograms computed from each block.

The lattice constant of the sub-arrays can alternatively be determined from the 2-D auto-correlation function of the micro-array. Starting from the origin of the 2-D auto-correlation function, the first peak that lies within a few degrees of the +x direction is identified. The distance from the origin to this peak is equal to the lattice constant in the x direction, and the orientation of the peak is the orientation of the sub-array. A similar measurement can be used to determine the lattice constant in the y direction. Because of the averaging nature of the auto-correlation function, the noise level of this analysis is low, diminishing the possibility of false peaks. Although the peak produced by the auto-correlation function may be broad, the step of fitting a curving surface around the peaks and calculating the apex of the surface can be used to estimate accurately the location of each peak. In the case of images with very high levels of noise and irregularity in the dot shape and placements, the peaks can be so broad that some of the peaks merge into the primary peak at the origin and become undetectable. In this case, the application of a smoothing function with an automatically calculated threshold may sufficiently enhance the image that peaks produced by the auto-correlation function become distinct.

Once the lattice constant and the orientation of each sub-array of the micro-arrays are known, the image can be rotated at step 206 so that the sides of the image align with the direction of the rows and columns of the micro-array. From this point, it is assumed that any misalignment between the x-axis and the rows of the sub-arrays, and the y-axis and the columns of the sub-array are negligible.

A sub-array image typically covers an area on the order of a few hundred thousand pixels. As such, a few hundred thousand pixels in the vicinity of each location in the image must be examined to determine if the pixels represent a structure resembling a sub-array. A template sub-array is created at step 208 that has the number of expected rows and columns and containing identical round dots spaced according to the estimated lattice constant. At step 210, the template sub-array is used as a guide to identify regions of the image that resemble sub-arrays. The identical round dots may each have a Gaussian profile. If a region is found in the image that resembles the template, it is likely that the region is a sub-array. The degree of resemblance between the region of the image and the template can be measured by the cross-correlation between the template and the region of the image. Following the application of the cross-correlation function between the region of the image and the template, and assuming that the origin of the template is at the geometrical center of the template, each local maximum point of the cross-correlated image represents a possible location of the geometrical center of a sub-array. The number of such possible locations can be reduced if each local maximum point is discarded when it is not the absolute maximum point in an area of a few grid cells around it.

If fewer possible sub-array regions are found, as compared to the number of expected sub-arrays, it is possible that the image has a strong background that drifts in amplitude from one side of the image to the other. To account for this, an automatically determined locally varying threshold function can be applied to the image before the cross-correlation function is computed. As a second alternative, a two-dimensional low-cut filter can be applied to the image to remove large-scale trends from the image. By automatically trying different combinations of these or other enhancements, an algorithm can be found that will locate the sub-arrays in all but the most problematic images, which must be visually examined by the user.

When more possible images are detected than there are sub-arrays, geometrical restrictions can be applied to exclude the false possible images. Possible geometric conditions include the rule of including sub-arrays that do not overlap and that are organized in approximately rectangular grids, the allowable regularity of which can be progressively restrained until only one set of conforming selections is left. As a second possible condition, the geometrical constraints can be relaxed, revealing multiple sets of conforming locations. Of these multiple sets of conforming locations, the set with the largest amplitude sum is selected. If these two conditions—restraining and relaxing the geometrical constraints—do not produce the same results, the user is alerted while the program proceeds with the result from the restraining methods.

After the location of each of the sub-arrays is determined, the expected location of each individual dot can be estimated on the basis of the estimated values of the lattice constant. The procedure for locating the sub-arrays of the image can be modified slightly to take advantage of added location information for those micro-array images that have two color channels. In the case of a micro-array image that includes two color channels, the process of locating the sub-arrays is first applied to one color channel, treating this channel as though it were a single channel gray-scale image. If this step fails to locate the sub-arrays, the procedure is repeated with the other color channel as the input. If this step continues to yield no definite result, the two channels are summed together to form a single gray scale image to be used as the input to the steps of locating the sub-arrays of the image.

### II. Refining Dot Placements

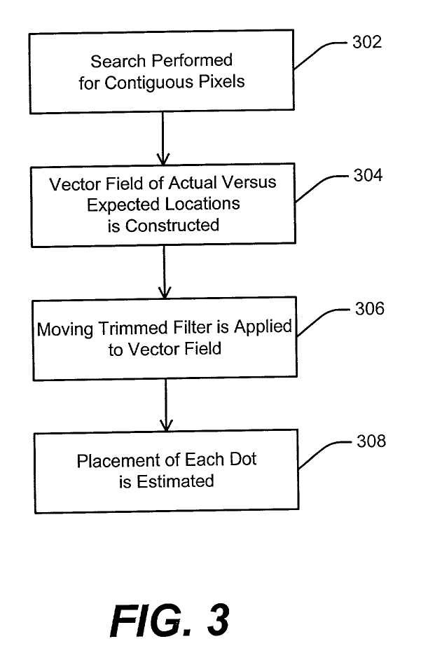

In some micro-arrays, the positions of the dots of the micro-array differ visibly from the expected location of the dots if the dots were to fall in place on a regular rectangular grid. For the purpose of forming the constraining shape mask, which is described later herein, the deviations and the actual centers of the dots are detected. Because of the presence of deviations in the dots and the possible presence of unknown artifacts in the image, it may not be possible to detect every dot in the micro-array. The method described herein searches for and accepts only those objects that look like dots. A flow diagram of the method is shown in FIG. 3.

Starting from the center of a grid point within a sub-array, an outward concentric search is performed for any objected formed by contiguous pixels that have intensities that are significantly greater than the background level of the image, as indicated by step 302 of FIG. 3. A search is performed for objects that are approximately round and of a size that is larger than a few pixels but smaller than the area of the largest circle that can be fit in a grid cell. In many cases, fewer qualified objects can be found for each sub-array than there are dots in the sub-array. A twenty percent (20%) trimmed mean of the area of all such objects is calculated, and any object whose area differs by more than a factor of two from this value is rejected. In performing this analysis, the requirement that neighboring dots should not overlap or touch is not imposed.

At step 304, for objects that satisfy the conditions of the outward concentric search, the displacements of the expected positions to their actual positions are calculated, forming for each sub-array a two-dimensional vector field. The vector field will include gaps in the data for those gaps where the displacement is undetermined. A two-dimensional moving trimmed filter is applied to each component of the field as part of step 306, and gaps in data of the field are filled with data achieved by interpolation. Applying these displacements to the regular grid positions yields an estimate for the position of every dot, as indicated by step 308 of FIG. 3. For images that are two-channel images, a displacement field is calculated on the basis of and from each channel. The channel that yields the more acceptable objects is used as the final result.

### III. Target Detection and Delineation

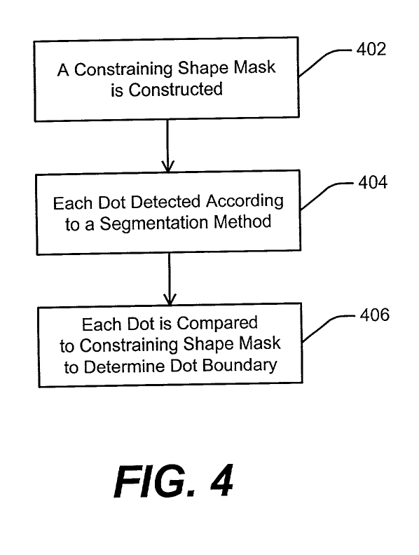

Some dots in the image may suffer from “bleed-over,” which is characterized by a dot that lacks a fully enclosed and clearly defined boundary. A constraining shape mask can be applied to impose a restraint on the maximum allowable size for a dot. A constraining mask is formed for each sub-array so that depositing pins with different physical characteristics can deposit one or more of the sub-arrays of the micro-array. The step of constructing a constraining shape mask is shown as step 402 in FIG. 4, which depicts the steps of delineating and detecting each dot of each sub-array of the micro-array. The shape mask is constructed from a stacked or summed image of the strongest of the dots in the sub-array. To determine the relative strength of a dot, a measurement can be made of the average intensities of the pixels within the grid cell. For this calculation, the grid cell of a dot is the rectangular area centered at the dot and with sides equal to the lattice constants. For the purpose of this analysis, the strongest dots of the sub-array can be considered to be the strongest 10% of the dots of the sub-array. As an alternative, the strongest dots of the sub-array can be considered to be all those dots above a certain threshold, with the threshold being calculated from an estimation of the background mean and variance. The dots that are determined to be strongest dots of the array are stacked together and summed.

Because the centers of the dots have not been determined with certainty, the dots may be misaligned when stacked, causing the summed image to be slightly blurry. The process of summing the images will also reduce the relative strength of the background noise variance. From the summed image, a threshold can be computed by, for example, the method disclosed by Otsu's method, which is described in Otsu, N., “A Threshold Selection Method from Gray-Level Histograms”, IEEE Transactions on Systems, Man, and Cybernetics, 9(1): 62-66, which is incorporated by reference herein. The shape mask is constructed by dilating by one pixel the object formed by the pixels above the threshold in the stacked image. The blurriness of the summed images and the dilation operation cause the shape mask to be larger than most dots in the array. If a dot that is detected in a later step does not fit within the shape mask, it is likely that the dot suffers from bleed-over, and the extent of the dot should be constrained to stay within the mask to prevent inclusion of background pixels into signal strength calculations. For two-channel images, strong dots from both channels can be used to produce the stacked image.

As part of step 404, each dot is detected according to a segmentation method. A threshold based segmentation method may be used to detect individual dots. For each dot to be detected, Otsu's method is used to compute a threshold from the histogram of the pixels within the dot's grid cell after the application of a median smoothing function to the image. The computed threshold is constrained to fall within a range determined from an estimate of the mean of the local background and variance. This background estimation is made on the basis of pixels that are outside the largest circles that can fit within the grid cells. Background pixels from a 5-by-5 grid cell area around each target dot are included for the analysis of that target dot. As an example, with a lattice constant of 25 pixels, there are about three thousand pixels included in the background estimation for each target dot. It has been found that the range of (μ+3σ, μ+10σ) to be a reasonable choice as a constraint on the threshold selected by Otsu's method, where μ and σ are the mean and variance, respectively, of the background.

Segementation methods that are based solely on the threshold analysis rely on the intensity of a pixel when determining whether the pixel represents a signal or background. These methods can be augmented by morphological operations that merge unconnected regions and smooth the contours of detected objects based on positional information. The hysteresis threshold method is a segmentation method that includes both an analysis of pixel intensity and morphological operations. The hysteresis threshold method uses a lower threshold for pixels that are connected to pixels above a higher threshold. The application of a constrained Otsu threshold followed by some minimal amount of morphological operation can achieve better results for most micro-array images, as compared with the application of a segmentation method that involves only intensity analysis.

The detected object is compared against the constraining shape mask, as indicated by step 406 of FIG. 4. When the object does not fit entirely within the mask, a best fit may be found by slightly sliding the mask around the area of the object. When a best fit is found, pixels that fall outside the boundary of the constraining shape mask are dropped. For two-channel images, the union of the two objects detected in the two channels are used as the final result, thereby reflecting the fact that the distributions of the two types of dyed genetic material that generate the image in each channel could differ. Because a dot is defined to be the area occupied by material deposited on the micro-array, the presence of one type of dyed material in a certain region is sufficient indication that the region is a part of a dot. For this reason, calculation of signal strength for either channel is carried out over all pixels in the union.

In addition to the noise generated by the random variation of the background image, confetti-like noise caused, for example, by large particles of contamination may also be present on the image. Such artifacts, typically in the form of a small group of high intensity pixels, are excluded from any signal calculations. On the basis of the assumption that all normal pixels belonging to a single dot are relatively homogeneous in terms of intensity, statistical outliers can be separated from normal signal pixels. The separation of the outlying artifacts can be accomplished by a second segmentation process that segments out pixels that are included as part of the signal during the first segmentation process. For two-channel images, any pixel detected as noise in either channel must be excluded from the signal calculations in both channels.

After the boundary of each dot is determined, signal and background statistics can be determined for each dot. Because the signal pixel intensities do not follow any simple random process model, a trimmed mean is the most appropriate measure of an estimate average intensity. The background pixel intensities can be modeled by a gamma distribution, although artifacts and other statistical outliers must first be excluded before the fitting of the gamma distribution. Background pixels from neighboring grid cells are typically included in the computation for each dot. Quality metrics that measure the signal against the background image, the shape and strength of a dot against the shape and strength of other dots in the array, and the local background against the background in other regions may also be applied to the dot computation.

The method disclosed herein is an automated technique for precise coordinate location in the x and y direction of each element in a micro-array. The method may be automated such that the method can be used as a means for the efficient processing of hundreds of images having within each thousands of individual dots.

Although the present disclosure has been described in detail, it should be understood that various changes, substitutions, and alterations can be made hereto without departing from the spirit and the scope of the invention as defined by the appended claims.

What is claimed is:

1. A method for identifying the individual elements of a micro-array, comprising the steps of:
calculating the lattice constant of the micro-array;
aligning a grid to a set of elements of the micro-array;
performing a local spatial adjustment to each element of the micro-array; and
delineating the boundaries of each grid element.

2. The method for identifying the individual elements of a micro-array of claim 1, wherein the step of calculating the lattice constant comprises the steps of:
performing a two-dimensional periodogram of the image; and measuring the distance between the two strongest peaks of the periodogram.

3. The method for identifying the individual elements of a micro-array of claim 2, wherein the step of calculating the lattice constant comprises the step of measuring the angle formed by a vector from the coordinates origin to the closest peak provides the angle of rotation of the grid array.

4. The method for identifying the individual elements of a micro-array of claim 3, wherein step of aligning the grid according to a set of elements of the micro-array comprises the step of aligning a grid of a sub-array of the micro-array according to the angle formed by a vector from the coordinates origin to the closest peak provides the angle of rotation of the grid array.

5. The method for identifying the individual elements of a micro-array of claim 1, wherein the step of calculating the lattice constant comprises the steps of:
applying an auto-correlation function to a sub-array of the micro-array;
identifying the first peak that lies in the x direction from the coordinates origin of the sub-array to calculate the lattice constant in the x direction;
identifying the first peak that lies in the y direction from the coordinates origin of the sub-array to calculate the lattice constant in they direction;

6. The method for identifying the individual elements of a micro-array of claim 5, wherein the step of calculating the lattice constant comprises the step of measuring the angle formed by a vector from the coordinates origin to the closest peak in the x direction.

7. The method for identifying the individual elements of a micro-array of claim 6, wherein step of aligning the grid according to a set of elements of the micro-array comprises the step of aligning a grid of a sub-array of the micro-array according to the angle formed by a vector from the coordinates origin to the closest peak in the x direction.

8. The method for identifying the individual elements of a micro-array of claim 1, wherein the step of aligning a grid to a set of elements of the micro-array comprises the step of creating a template sub-array that includes identical round dots spaced from one another according to the lattice constant.

9. The method for identifying the individual elements of a micro-array of claim 8, wherein the step of aligning a grid to a set of elements of the micro-array comprises the step of cross-correlating the template sub-array against a selected set of elements of the sub-array.

10. The method for identifying the individual elements of a micro-array of claim 1, wherein the step of performing a local spatial adjustment to each element of the micro-array, comprises the step of refining the dot placement of each dot of each sub-array of the micro-array.

11. The method for identifying the individual elements of a micro-array of claim 10,
wherein the step of aligning a grid to a set of elements of the micro-array comprises the step of aligning a rectilinear grid to a sub-array of the micro-array; and
wherein the step of refining the dot placement of each dot of each sub-array of the micro-array comprises the step of, from the center of the grid, performing an outward concentric search for objects formed by contiguous pixels and having an intensity greater than the background of the image of the micro-array.

12. The method for identifying the individual elements of a micro-array of claim 11, wherein the step of refining the dot placement of each dot of each sub-array of the micro-array comprises the step of calculating for each dot the difference between the actual position and the expected position.

13. The method for identifying the individual elements of a micro-array of claim 12, wherein the step of refining the dot placement of each dot of each sub-array of the micro-array comprises the step of creating a vector filed identifying the distance and direction between the actual position and the expected position of each dot in the sub-array.

14. The method for identifying the individual elements of a micro-array of claim 1, wherein the step of delineating the boundaries of each grid element comprises the step of applying a constraining shape mask to each dot of each sub-array of the micro-array.

15. The method for identifying the individual elements of a micro-array of claim 14, wherein the step of applying a constraining shape mask to each dot of each sub-array of the micro-array comprises the step of summing the image of the dots in the sub-array having the highest intensity to form a constraining shape mask.

16. The method for identifying the individual elements of a micro-array of claim 15, wherein the step of applying a constraining shape mask to each dot of each sub-array of the micro-array comprises the steps of,
applying the constraining shape mask to each dot of the sub-array; and
disregarding any pixels that fall outside the boundary of the shape mask.

17. The method for identifying the individual elements of a micro-array of claim 16, wherein the step of applying a constraining shape mask comprises the step of adjusting the location of the constraining shape mask to achieve a fit of the shape mask to the dot of the sub-array.
